(19) United States
(12) Patent Application Publication
(10) Pub. No.: US 2002/0001804 A1
(43) Pub. Date: Jan. 3, 2002

(54) Genomic analysis of tRNA gene sets

(76) Inventors: Wayne Mitchell, San Francisco, CA (US); T. Guy Roberts, Oakland, CA (US)

Correspondence Address: LAW OFFICES OF JONATHAN ALAN QUINE, P O BOX 458, ALAMEDA, CA, 94501

(21) Appl. No.: 09/792,878

(22) Filed: Feb. 23, 2001

Related U.S. Application Data
(60) Provisional application No. 60/185,000, filed on Feb. 25, 2000.

(51) Int. Cl. C12Q 1/68; G06F 19/00; G01N 33/48; G01N 33/50

(52) U.S. Cl. 435/6; 702/20

(57) ABSTRACT

Methods for identifying one or more positions of conserved difference in a set of similar sequence strings are provided, as well as systems and devices for identifying one or more positions of conserved difference in a set of similar sequence strings, and sets of positions of conserved differences.

### CROSS-REFERENCE TO RELATED APPLICATIONS

This application is related to U.S. Ser. No. 60/185,000, filed Feb. 25, 2000; U.S. Ser. No. 60/185,071, also filed Feb. 25, 2000; U.S. Ser. No. 60/225,506, filed Aug. 15, 2000; and U.S. Ser. No. 60/225,505, also filed Aug. 15, 2000. The present application claims priority to, and benefit of, these applications pursuant to 35 U.S. C. §119(e).

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Molecular biology and drug discovery are in the midst of a profound transformation. The convenience and speed of automated experimental protocols, coupled with the extensive computational powers currently available, are generating an enormous amount of unrefined information. However, fairly sophisticated sets of computational tools are necessary to fully exploit the vast quantity of information gleaned thus far.

Algorithms and programs adapted for analyzing nucleic acid and/or protein sequence databases, and determining percent sequence identity and sequence similarity, are known in the art. One algorithm commonly used for sequence analysis is the BLAST algorithm, described in Altschul et al.(1990) J. Mol. Biol. 215:403-410, and publicly available from the National Center for Biotechnology Information (http://www.ncbi.nlm.nih.gov). The BLAST algorithm searches for similar sequence strings by first identifying relatively short strings within a first, or initial, sequence string, extending the similarity comparison (in both directions) along the discovered longer sequence strings (see, Altschul for a more detailed description). Typically, the short string used to initiate the search ranges in length from about three elements, for amino acid sequence searches, to around eleven elements for nucleotide sequence searches; however, these values can be adjusted based upon the desired search protocol. Determination of the percentage of sequence identity is inherent in the search protocol, since cumulative alignment scores are determined as an integral part of the algorithm during the search process. Cumulative scores are calculated for nucleotide sequences using “reward scores” for matching elements (having a value always greater than zero) and “penalty scores” for mismatching elements (often having values less than zero). For amino acid sequences, a more complicated scoring matrix, such as the BLOSUM62 scoring matrix is used to calculate the cumulative score (see Henikoff & Henikoff (1989) Proc. Natl. Acad. Sci. USA 89:10915). The BLAST algorithm also provides a statistical analysis of the similarity between two sequences (see, e.g., Karlin & Altschul (1993) Proc. Natl. Acad. Sci. USA 90:5873-5787). For example, the BLAST algorithm provides a calculation of the smallest sum probability (P(N)), a measure of similarity which indicates the probability that a match between two sequence strings would occur by chance.

Thus, the BLAST algorithm and other similar protocols are directed toward detection and analysis of similarities in sequence within sequence databases. The present invention provides alternative approaches to the analysis of sequence databases, as well as methods that can be used for discovering and assessing novel sites within sets of sequences that can be targeted for therapeutic interaction.

### SUMMARY OF THE INVENTION

The availability of genomic sequences for a variety of organisms provides, among other things, the opportunity to survey these genomes, or a derivative thereof, for multiple regions of homology. BLAST and other similar algorithms are useful for searching and analyzing such nucleic acid sequence databases, as well as protein sequence databases. However, these algorithms are directed toward, and consequently limited to, detection and analysis of similarities in structure. Perhaps as a result, it is often these similarities in structure that are employed when designing novel pharmaceuticals. However, similar sequence strings can contain specifically conserved regions of dissimilarity, such as the presence of conserved positions within a sequence string that accommodate dissimilar elements in order to impart specificity among members of a group of similar sequence strings. The presence of such positions is not detected by currently-available protocols and algorithms such as BLAST; rather, these dissimilar elements are most likely considered detrimental by such algorithms (i.e., the dissimilar elements are, by definition, not identical and thus decrease the degree of similarity between molecules). Thus, this relevant sequence information is not detected or analyzed using the algorithms available in the art, suggesting that alternative analytical approaches would be useful.

The present invention provides methods for identifying one or more positions of conserved difference in a set of similar sequence strings. The set of similar sequence strings, which are composed of at least n sequence elements, are derived from a plurality of species. Optionally, each species in the plurality of species contributes at least two similar sequence strings to the set. The methods include the steps of providing a set of similar sequence strings as described above; comparing the at least n sequence elements in a first similar sequence string to the at least n sequence elements in a second similar sequence string, for a first species of the plurality of species; assigning a value to each of n positions of the at least n sequence elements, based upon whether the sequence elements are identical or different in the two similar sequence strings; repeating the comparing and assigning for each species in the plurality of species; summing the values assigned for each of the n positions across the plurality of species; and identifying which of the n positions have the greatest sum value, thereby identifying the positions of conserved difference in the set of similar sequence strings.

The set of similar sequence strings can be acquired from a variety of species, including, but not limited to, prokaryotes (e.g., eubacterial species, archaea species) eukaryotes, and combinations thereof. Sets of similar sequence strings can be obtained by using one or more logical instructions (e.g., a computer-based searching algorithm) to search available sequences and identify the desired target sequences. The sequences to be analyzed can be amino acid sequences, nucleic acid sequences, carbohydrate sequences, and the like. In one embodiment of the present invention, the set of similar sequence strings are a set of tRNA sequences.

Optionally, the steps of comparing the sequence elements and assigning values to each position in the sequence is performed using a computer. In a further step, the positions that were determined to have the greatest sum value are assessed for their ability to interact with a cellular factor, such as a protein, a peptide, a protein complex, a nucleic acid, a protein-nucleic acid complex, a carbohydrate chain, or a combination of these factors. As one example, the position(s) identified by the methods of the present invention may interact with an enzyme at, for example, an active site or a regulatory site. As another example, the identified position(s) may interact with a protein-nucleic acid complex, e.g., a ribosome.

Furthermore, the methods of the present invention are not limited to a pairwise comparison of similar sequence strings. The aligned elements of three, four, ten, one hundred, or any number of sequence strings can be compared sequentially (e.g., pairwise) or simultaneously (e.g., higher order multiwise comparisons) using the described methods.

In addition, the methods of the present invention can further include the step of determining whether the identified position(s)of conserved difference have modified elements, for example, amino acids, nucleotides, or carbohydrate elements that have been changed or altered from their original or customary state (e.g., methylated, alkylated, acetylated, esterified, ubiquitinated, lysinylated, sulfated, phosphorylated, glycosylated, and the like).

Furthermore, the present invention provides a computer or computer readable medium having one or more logical instructions for identifying at least one conserved difference in a set of similar sequence strings derived from a plurality of species. In one embodiment, the computer or computer-readable medium employs logical instructions to compare at least n sequence elements in a first similar sequence string to at least n sequence elements in a second similar sequence string, for a first species of the plurality of species; assign a value to each of n positions of the at least n sequence elements, based upon whether the sequence elements are identical or different in the two similar sequence strings; repeat the comparing and assigning for each species in the plurality of species; sum the values assigned for each of the n positions across the plurality of species; and identify which of the n positions have the greatest sum value, thereby identifying the positions of conserved difference in the set of similar sequence strings.

The present invention also provides the set of conserved differences in a set of similar sequence strings, as identified by the methods, or using the computer or computer-readable medium, of the present invention. Furthermore, the present invention also provides compounds which interact at one or more of positions of conserved dissimilarity, as determined by the methods of the present invention.

The methods, compositions, and devices of the present invention provide novel mechanisms by which informational data, such as genomic sequences, can be analyzed. For example, using the methods of the present invention, a set of similar sequences of tRNA genes from eubacteria and archaea were analyzed to identify positions of conserved differences in nucleic acid sequence among species. Because the plurality of species, as exemplified by one embodiment, included representatives of divergent bacterial species, generalizations which emerge from comparative analysis of the set can be applied to other species, including those not present in the sample. Certain trends occur without exception in this sample and may be universal among prokaryotes. Furthermore, this information can be used in the design and assessment of pharmaceutical agents which will interact with a collective group, or with specified targets. The methods, compositions, and devices of the present invention can provide similar information from other sets of similar sequence strings, such as proteins sequences, carbohydrates structures involved in cellular adhesion or immune responses, and the like.

### BRIEF DESCRIPTION OF THE DRAWINGS

FIG. 1 is a flow chart illustrating a method for identifying one or more positions of conserved difference in a set of similar sequence strings according to an embodiment of the present invention.

FIG. 2 is a flow chart illustrating an alternative method for identifying one or more positions of conserved difference in a set of similar sequence strings according to another embodiment of the invention.

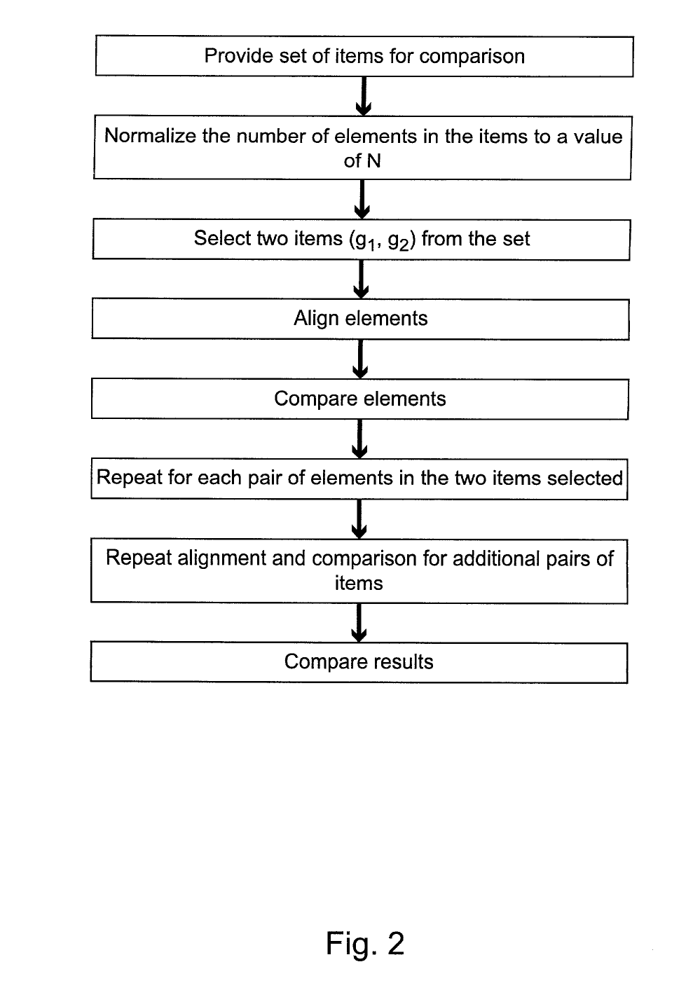

FIG. 3 is a flow chart illustrating an alternative method for identifying one or more positions of conserved difference in a set of similar sequence strings according to a further embodiment of the invention.

FIG. 4 is a pictorial representation of a computer or computer-readable medium of the present invention, in which the methods of present invention can be embodied.

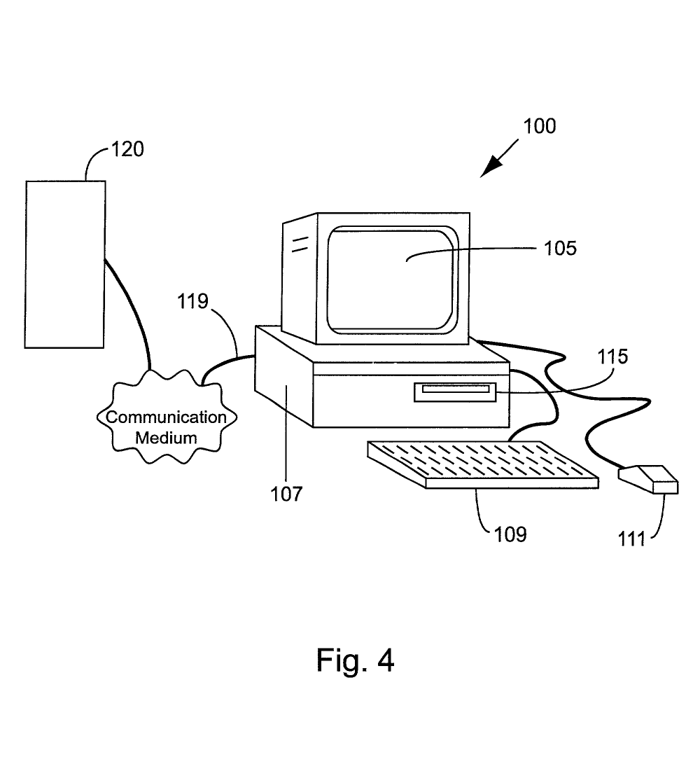

### DETAILED DISCUSSION OF THE INVENTION

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a similar sequence string” includes a combination of two or more such sequence strings, reference to “a tRNA molecule” includes mixtures of tRNA molecules, and the like.

Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

In describing and claiming the present invention, the following terminology will be used in accordance with the definitions set out below.

As used herein, the term “similar sequences string” refers to a series of arranged elements which are similar in element identity and in positional order to other series of arranged elements. The arranged elements can be nucleic acids, amino acids, sugar units, and the like. The degree of similarity between sequence strings can be calculated by a number of statistical methods available in the art; one common measure of similarity is, for example, determination of the smallest sum probability. For example, a nucleic acid sequence string can be considered similar to a reference sequence string if the smallest sum probability in a comparison of the test sequence string to the reference sequence string is less than about 0.1, or less than about 0.01, and or even less than about 0.001.

A “discriminatory position” in a similar sequence string is a position which has a extensive effect on the function of the entire molecule (e.g., the choice of element in this position plays a major role in establishing the function of the molecule).

The term “anticodon sequence” or “anticodon type” refers to the three nucleotides at positions 34, 35 and 36 in the tRNA structure, that interacts with the codon region of a MRNA molecule during the process of translation. An anticodon sequence is described as “censored” if it does not occur in the plurality of genomes examined. An anticodon sequence is described as “under-represented” if it occurs in about fifty percent or fewer of the plurality of genomes.

A “tRNA type” of a tRNA molecule is defined by the anticodon sequence of the tRNA molecule, as predicted from the DNA sequence of the corresponding gene. There are 64 potential triplet codons; three “stop” codons and 61 codons that can encode the twenty amino acids (and therefore, there are potentially 61 different tRNA types).

The term “species” as used herein refers to members of a group of similar items. In one context, the term is used to refer to the taxonomic categories delineated under the Linnean genus/species naming convention. The bacterial species Escherichia coli, Haemophilus influenzae, and Helicobacter pylori are example of this context. In other contexts, the term species is used to refer to sets of items similar in at least one particular or defined feature, but not necessarily biological organisms, e.g., of the Linnean system of classification. An example of this alternate use of the term is depicted when referring to the automotive “species” of Ford Mustang, Dodge Viper, and Toyota Celica. As another example, the general species of “cars” can be considered, distinct from other transportation vehicles such as delivery vans, trucks, or buses. Other examples, such as races of people, populations of cities, groups of astronomical bodies, and other items that are considered as a group or set for the purpose of analysis, would be recognized as “species” by one of skill in the art.

In Silico Discovery of Therapeutic Targets

Pharmaceutical companies are pursuing new drug targets by a variety of in vitro and in vivo based experimental methods, including random screening of collections of genes against compound libraries. An alternative approach to this “wet chemistry” approach to discovery of potential therapeutic targets is in silico, or theoretical calculation/molecular modeling-based identification of interesting (i.e. potentially targetable) structural and/or functional regions within a set of structurally-related molecules. Customarily, this analytical approach searches for regions of conserved structure among related molecules, and, as such, is the basis for “rational drug design” approaches to drug discovery. Changes to conserved regions in the molecule generally lead to loss of activity or another desired characteristic. Therefore, regions of dissimilarity would not be expected to yield novel sites of pharmaceutical interaction. Thus, it is a unique approach to survey a set of similar structures for regions in which they regularly differ in structure, rather than regions of constancy, and as shown herein, this approach can unexpectedly be used to identify novel sites for therapeutic action.

The present invention provides methods for identifying one or more positions of conserved difference in a set of similar sequence strings, as well as the sets of conserved differences, and systems and devices to identify these sites. The set of similar sequence strings used in the methods of the present invention are composed of at least n sequence elements, and are derived from a plurality of species. Because the plurality of species can include a variety of divergent representatives, the methods of the present invention can provide generalizations that may be applicable to multiple species, including those not present in the sample. The extent of divergence in the positions of conserved difference can be used to tailor therapeutic agents toward specific species, versus general, nonspecies-specific interactions.

In one embodiment of the present invention, the comparative analysis of the transfer RNA (tRNA) gene sets from eighteen bacterial genomes was undertaken, and a number of sites of conserved differences were identified. The occurrence of tRNA gene types is highly biased within the eighteen bacterial species currently available for analysis. Some of the patterns of tRNA gene type frequency appear to be universal among bacterial species.

Similar Sequence Strings

The similar sequences strings to be analyzed in the methods of the present invention can be composed of a number of elements, such as amino acids, nucleic acids, carbohydrates, and the like. Each similar sequence string has at least n sequence elements to be analyzed for positions of conserved differences; as such, the positions of the at least n elements are aligned with each other based upon the homology, prior to performing the analysis. Thus, the two or more similar sequence strings to be analyzed need not contain the same number of elements; in sets where the number of elements differ, only those portions of the sequence strings having corresponding elements are analyzed.

The sets of similar sequence strings employed in the methods and compositions of the present invention can be acquired from a variety of sources, including, but not limited to laboratory sequencing results; published records; public and/or private databases, such as those listed with the National Center for Biotechnology Information (www.ncbi.nlm.nih.gov) in the GenBank® databases; sequences provided by other public or commercially-available databases (for example, the NCBI EST sequence database, the EMBL Nucleotide Sequence Database, Incyte's (Palo Alto, Calif.) LifeSeq™ database, and Celera's (Rockville, Md.) “Discovery System”™ database); Internet listings, and the like.

The similar sequence strings can be derived from a plurality of species, including, but not limited to, prokaryotes, eukaryotes, and combinations thereof. Furthermore, the similar sequence strings can be derived from a plurality of prokaryotic species, including, but not limited to, eubacterial species, archaea species, and combinations thereof. Eubacterial species include, but are not limited to, hydrogenobacteria, thermatogales, deinococcus, cyanobacteria, purple bacteria, green sulfur bacteria, green non-sulfur bacteria, planctomyces, spirochetes, cytophages, flavobacteria, bacteroides, and gram positive bacteria. Archaebacteria include, but are not limited to, methanogens, extreme thermophiles, and extreme halophiles. (See, for example, the lists of microorganism genera provided by DSMZ-Deutsche Sammlung von Mikroorganismen und Zellkulturen GmbH, Braunschweig, Germany, at http://www.dsmz.de/species.) A noncomprehensive list of exemplary species for use in the methods of the present invention can be found in Tables 1 and 2. Furthermore, the plurality of species can be comprised of non-taxonomical species, such as populations of people, sets of car makes and models, astronomical bodies, or any group of items to be analyzed. Preferably, each species contributes at least two similar sequence strings to the set of similar sequence strings to be analyzed. Optionally, multiple similar sequence strings can be contributed. Furthermore, the multiple similar sequence strings can be compared in a pairwise manner (e.g., sequentially), or in grouped sets, or simultaneously as a whole (a higher order comparison).

In one embodiment, the set of similar sequence strings employed in the methods of the present invention are a set of tRNA sequences. The tRNA sequences are defined by the anticodon sequence carried by the tRNA gene. There are 61 triplet codons that encode the twenty amino acids (and three codons that encode “stop” signals). Therefore, there are potentially 61 different tRNA types. See, for example, Lehninger (1982) Principles of Biochemistry (Worth Publishers, Inc., New York). Table 1 provides a listing the 64 possible DNA codons (including the three stop codons, one of which, TGA, sometime encodes selenocysteine), the 64 tRNA anticodon types, the corresponding amino acid, and the tRNA frequencies from each bacterial genome by type.

TABLE 1
FREQUENCY OF TRNA ANTICODONS IN SELECTED MICROBIAL
GENOMES

| Amino | | Anti | | | | | | | | | | | | | | | | | | |
|---|---|---|---|---|---|---|---|---|---|---|---|---|---|---|---|---|---|---|---|---|
| acid | Codon | codon | Mg | Mp | Ct | Rp | Tp | Cp | Bb | Aa | Hp | Mj | Mt | Ph | Hi | Af | Sy | Bs | Tb | Ec |
| F | TTT | aaa | 0 | 0 | 0 | 0 | 0 | 0 | 0 | 0 | 0 | 0 | 0 | 0 | 0 | 0 | 0 | 0 | 0 | 0 |
| | TTC | gaa | 1 | 1 | 1 | 2 | 1 | 1 | 2 | 1 | 1 | 1 | 1 | 1 | 1 | 1 | 1 | 1 | 1 | 2 |
| L | TTA | uaa | 1 | 1 | 1 | 1 | 1 | 1 | 1 | 1 | 1 | 1 | 1 | 1 | 2 | 1 | 1 | 3 | 0 | 1 |
| | TTG | caa | 1 | 1 | 1 | 0 | 0 | 1 | 0 | 1 | 1 | 0 | 0 | 1 | 1 | 1 | 0 | 0 | 1 | 1 |
| S | TCT | aga | 0 | 0 | 0 | 0 | 0 | 0 | 0 | 0 | 0 | 0 | 0 | 0 | 0 | 0 | 0 | 2 | 1 | 0 |
| | TCC | gga | 1 | 1 | 1 | 1 | 1 | 1 | 1 | 1 | 1 | 1 | 2 | 1 | 1 | 1 | 1 | 1 | 1 | 3 |
| | TCA | uga | 1 | 1 | 1 | 1 | 1 | 1 | 1 | 1 | 1 | 1 | 1 | 1 | 2 | 1 | 1 | 2 | 1 | 1 |
| | TCG | cga | 1 | 2 | 1 | 0 | 1 | 1 | 0 | 1 | 0 | 0 | 0 | 1 | 0 | 1 | 1 | 0 | 1 | 1 |
| Y | TAT | aua | 0 | 0 | 0 | 0 | 0 | 0 | 0 | 0 | 0 | 0 | 0 | 0 | 0 | 0 | 0 | 0 | 0 | 0 |
| | TAC | cua | 1 | 1 | 1 | 1 | 1 | 1 | 0 | 1 | 1 | 1 | 1 | 1 | 1 | 1 | 1 | 2 | 1 | 2 |
| stop | TAA | uua | | | | | | | | | | | | | | | | | | |
| stop | TAG | gua | | | | | | | | | | | | | | | | | | |
| C | TGT | aca | 0 | 0 | 0 | 0 | 1 | 0 | 1 | 0 | 1 | 0 | 0 | 0 | 0 | 0 | 1 | 1 | 0 | 0 |
| | TGC | gca | 1 | 1 | 1 | 1 | 1 | 1 | 1 | 1 | 0 | 1 | 1 | 1 | 1 | 1 | 1 | 1 | 1 | 1 |
| Stop 1 | TGA | uca | S | S | | | | | | S | | S | | | | | | | | S |
| W | TGG | cca | 1 | 1 | 1 | 1 | 1 | 1 | 1 | 1 | 1 | 1 | 1 | 1 | 2 | 1 | 1 | 1 | 1 | 1 |
| L | CTT | aag | 0 | 0 | 0 | 0 | 0 | 0 | 0 | 0 | 0 | 0 | 0 | 0 | 0 | 0 | 0 | 0 | 0 | 0 |
| | CTC | gag | 1 | 1 | 1 | 1 | 0 | 1 | 1 | 1 | 1 | 1 | 1 | 1 | 1 | 1 | 0 | 1 | 1 | 1 |
| | CTA | uag | 1 | 1 | 1 | 1 | 0 | 1 | 1 | 1 | 1 | 1 | 1 | 1 | 1 | 1 | 1 | 2 | 1 | 1 |
| | CTG | cag | 0 | 0 | 1 | 0 | 1 | 1 | 0 | 1 | 0 | 0 | 0 | 1 | 0 | 1 | 1 | 1 | 1 | 4 |
| P | CCT | agg | 0 | 0 | 0 | 0 | 0 | 0 | 0 | 0 | 0 | 0 | 0 | 0 | 0 | 0 | 1 | 0 | 0 | 0 |
| | CCC | ggg | 0 | 0 | 1 | 0 | 1 | 1 | 0 | 1 | 1 | 1 | 0 | 1 | 0 | 1 | 1 | 0 | 1 | 1 |
| | CCA | ugg | 1 | 1 | 1 | 1 | 1 | 1 | 1 | 1 | 1 | 1 | 1 | 2 | 1 | 1 | 1 | 3 | 1 | 1 |
| | CCG | cgg | 0 | 0 | 0 | 0 | 1 | 0 | 0 | 1 | 0 | 0 | 0 | 1 | 0 | 1 | 1 | 0 | 1 | 1 |
| H | CAT | aug | 0 | 0 | 0 | 0 | 0 | 0 | 0 | 0 | 0 | 0 | 0 | 0 | 0 | 0 | 0 | 0 | 0 | 0 |
| | CAC | gug | 1 | 1 | 1 | 1 | 1 | 1 | 1 | 1 | 1 | 1 | 1 | 1 | 1 | 1 | 0 | 2 | 1 | 1 |
| Q | CAA | uug | 1 | 1 | 1 | 1 | 1 | 1 | 1 | 1 | 1 | 1 | 1 | 1 | 2 | 1 | 1 | 4 | 1 | 2 |
| | CAG | cug | 0 | 0 | 0 | 0 | 1 | 0 | 0 | 0 | 0 | 0 | 0 | 0 | 0 | 1 | 1 | 0 | 1 | 2 |
| R | CGT | acg | 0 | 0 | 1 | 1 | 1 | 1 | 0 | 1 | 0 | 0 | 0 | 0 | 2 | 1 | 1 | 4 | 1 | 4 |
| | CGC | gcg | 1 | 1 | 0 | 0 | 1 | 0 | 1 | 0 | 1 | 1 | 1 | 1 | 0 | 1 | 0 | 0 | 0 | 0 |
| | CGA | ucg | 1 | 1 | 1 | 0 | 1 | 1 | 1 | 0 | 1 | 1 | 1 | 1 | 0 | 1 | 0 | 0 | 0 | 0 |
| | CGG | ccg | 0 | 0 | 0 | 1 | 1 | 0 | 0 | 1 | 0 | 0 | 0 | 1 | 1 | 1 | 1 | 1 | 1 | 1 |
| I | ATT | aau | 0 | 0 | 0 | 0 | 0 | 0 | 0 | 0 | 0 | 0 | 0 | 0 | 0 | 0 | 0 | 0 | 0 | 0 |
| | ATC | gau | 1 | 1 | 1 | 1 | 1 | 1 | 1 | 2 | 1 | 1 | 1 | 1 | 3 | 1 | 1 | 3 | 1 | 3 |
| | ATA | uau | 0 | 0 | 0 | 0 | 0 | 0 | 0 | 0 | 0 | 0 | 0 | 0 | 0 | 0 | 0 | 0 | 0 | 0 |
| M | ATG | cau | 3 | 3 | 3 | 3 | 3 | 3 | 3 | 3 | 3 | 3 | 3 | 3 | 3 | 3 | 3 | 5 | 3 | 8 |
| T | ACT | agu | 0 | 0 | 0 | 0 | 0 | 0 | 0 | 0 | 0 | 0 | 0 | 0 | 0 | 0 | 0 | 0 | 0 | 0 |
| | ACC | cgu | 1 | 1 | 1 | 1 | 1 | 1 | 1 | 1 | 1 | 1 | 1 | 1 | 1 | 1 | 1 | 1 | 1 | 2 |
| | ACA | ugu | 1 | 1 | 1 | 1 | 1 | 1 | 1 | 1 | 1 | 1 | 1 | 1 | 1 | 1 | 1 | 4 | 1 | 1 |
| | ACG | cgu | 1 | 1 | 1 | 1 | 1 | 1 | 0 | 1 | 0 | 0 | 1 | 1 | 0 | 1 | 1 | 0 | 1 | 1 |
| N | AAT | auu | 0 | 0 | 0 | 0 | 0 | 0 | 0 | 0 | 0 | 0 | 0 | 0 | 0 | 0 | 0 | 0 | 0 | 0 |
| | AAC | guu | 1 | | 1 | 1 | 1 | 1 | 1 | 1 | 1 | 1 | 1 | 1 | 2 | 1 | 1 | 4 | 1 | 3 |
| K | AAA | uuu | 1 | 1 | 1 | 1 | 1 | 1 | 1 | 1 | 1 | 1 | 1 | 1 | 3 | 1 | 1 | 4 | 1 | 6 |
| | AAG | cuu | 1 | 1 | 0 | 0 | 1 | 0 | 1 | 1 | 0 | 0 | 0 | 1 | 1 | 1 | 0 | 0 | 1 | 0 |
| S | AGT | acu | 0 | 0 | 0 | 0 | 0 | 0 | 0 | 0 | 0 | 0 | 0 | 0 | 0 | 0 | 0 | 0 | 0 | 0 |
| | AGC | gcu | 1 | 1 | 1 | 1 | 1 | 1 | 1 | 1 | 1 | 1 | 1 | 1 | 1 | 1 | 1 | 2 | 1 | 1 |
| R | AGA | ucu | 1 | 1 | 1 | 1 | 1 | 1 | 1 | 1 | 1 | 1 | 1 | 1 | 1 | 1 | 1 | 1 | 1 | 1 |
| | AGG | ccu | 1 | 1 | 0 | 0 | 1 | 1 | 0 | 1 | 1 | 0 | 1 | 1 | 0 | 1 | 1 | 1 | 1 | 1 |
| V | GTT | aac | 0 | 0 | 0 | 0 | 0 | 0 | 0 | 0 | 0 | 0 | 0 | 0 | 0 | 0 | 0 | 0 | 0 | 0 |
| | GTC | gac | 0 | 0 | 1 | 0 | 1 | 1 | 0 | 1 | 1 | 1 | 1 | 1 | 1 | 1 | 1 | 1 | 1 | 2 |
| | GTA | uac | 1 | 1 | 1 | 1 | 1 | 1 | 1 | 1 | 1 | 1 | 1 | 1 | 1 | 1 | 1 | 4 | 1 | 5 |
| | GTG | cac | 0 | 0 | 0 | 0 | 1 | 0 | 0 | 0 | 0 | 1 | 1 | 1 | 0 | 2 | 0 | 0 | 1 | 0 |
| A | GCT | agc | 0 | 0 | 0 | 0 | 0 | 0 | 0 | 0 | 0 | 0 | 0 | 0 | 0 | 0 | 0 | 0 | 0 | 0 |
| | GCG | ggc | 0 | 0 | 1 | 0 | 1 | 1 | 0 | 1 | 1 | 1 | 1 | 1 | 1 | 1 | 1 | 1 | 1 | 2 |
| | GCA | ugc | 1 | 1 | 1 | 1 | 1 | 1 | 1 | 2 | 1 | 2 | 2 | 1 | 2 | 1 | 1 | 5 | 1 | 2 |
| | GCG | cgc | 0 | 0 | 0 | 0 | 1 | 0 | 0 | 0 | 0 | 0 | 0 | 0 | 0 | 0 | 0 | 0 | 1 | 0 |
| D | GAT | auc | 0 | 0 | 0 | 0 | 0 | 0 | 0 | 0 | 0 | 0 | 0 | 0 | 0 | 0 | 0 | 0 | 0 | 0 |
| | GAC | guc | 1 | 1 | 1 | 1 | 1 | 1 | 1 | 1 | 1 | 1 | 1 | 1 | 2 | 1 | 1 | 4 | 1 | 3 |
| E | GAA | uuc | 1 | 1 | 1 | 1 | 0 | 1 | 0 | 1 | 2 | 2 | 1 | 1 | 3 | 1 | 1 | 5 | 1 | 4 |
| | GAG | cuc | 0 | 0 | 0 | 0 | 1 | 0 | 0 | 0 | 0 | 0 | 0 | 1 | 0 | 0 | 0 | 0 | 1 | 0 |
| G | GGT | acc | 0 | 0 | 0 | 0 | 0 | 0 | 0 | 0 | 0 | 0 | 0 | 0 | 0 | 0 | 0 | 0 | 0 | 0 |
| | GGC | gcc | 1 | 1 | 1 | 1 | 1 | 1 | 1 | 1 | 1 | 1 | 1 | 1 | 3 | 1 | 1 | 4 | 1 | 4 |
| | GGA | ucc | 1 | 1 | 1 | 1 | 1 | 1 | 1 | 1 | 1 | 1 | 1 | 1 | 1 | 1 | 1 | 3 | 1 | 1 |
| | GGG | ccc | 0 | 0 | 0 | 0 | 0 | 0 | 0 | 1 | 0 | 0 | 0 | 1 | 0 | 0 | 1 | 0 | 1 | 1 |

Note:
the abbreviations for the bacterial species listed in the column header are given in Table 2.

Method of Identifying Positions of Conserved Differences

The present invention provides methods for identifying one or more positions of conserved difference in a set of similar sequence strings. The methods starts with providing a set of similar sequence strings as described above. Next, the at least n sequence elements in a first similar sequence string are compared to the at least n sequence elements in a second similar sequence string, for a first species of the plurality of species. The two similar sequence strings from the species are considered a “sib-pair,” reflecting their similarity in sequence and in origin.

Alternatively, each of the sequence elements in multiple (e.g., more than two) similar sequence strings from a given species are compared simultaneously, or in groups of more than two (i.e., a higher order comparison rather than a pairwise comparison). The multiple similar sequence strings from the species are considered a “sib-multiplet,” reflecting their higher order state as compared to a “sib-pair” as well as the similarity in sequence and in origin.

A value is assigned to each of n positions of the at least n sequence elements, based upon whether the sequence elements are identical or different in the two (or more) similar sequence strings. While any value can be used in this calculation, preferably a value of “one” is assigned to positions having different elements, and a value of “zero” is assigned to positions having the same element. When performing higher order analyses, the value can be greater than one, and optionally would reflect the number of differences noted among the multiple similar sequence strings being analyzed. In either of these embodiments of the methods of the present invention, any elements present in the sequence string but in excess of (i.e. outside) the n paired elements are optionally not considered in the calculation.

Optionally, the comparing of the n elements in the sib-pair (or sib-multiplet) and assigning values to each position in the sequence is performed using a computer. In one embodiment of the methods of the present invention, this process of comparing and assigning is repeated for each sib-pair in the species (if more than two sequence strings are present) and for each species in the plurality of species. The values assigned for each of the n positions across the plurality of species are then summed together, to provide a numeric value for each position. Using the valuation described above, the sum can range from zero (for positions in which the element is always the same regardless of species) to a maximum value equal to the number of sib-pairs or sib-multiplets examined in the plurality of species (in cases in which none of the elements are identical across species).

Finally, the positions having the greatest sum value are determined, thereby identifying positions of conserved difference in the set of similar sequence strings. This process is termed “disjunction analysis.” Variation in the identity of elements between sib-pairs suggests that these positions can represent functionally important features, such as “discriminatory positions.”

Discriminatory positions are important in defining the functional divergence of similar but non-identical molecules, such as pairs of protein paralogs with divergent biochemical activities, or, for example, distinct tRNA subtypes. For tRNA molecules, a discriminatory position can be characterized as follows. Two related tRNA molecules, such as two different elongator tRNA molecules, are compared base for base, starting at position one and proceeding through the tRNA sequence to position seventy-three. Alternatively, the genes encoding the tRNA sequences can be compared. Positions having non-identical elements are assigned a value of one, while positions having identical elements are assigned a value of zero. For example, in Bacterium sp., if elongator tRNA-1 is compared to elongator tRNA-2, and at position 2 the base “g” occurs in elongator tRNA-1 and the same base, a “g” occurs in elongator tRNA-2, then the position 2 is scored “zero” in that genome. At position three, tRNA-1 might be “a”, while tRNA-2 might be “g”. This is a “discriminatory position” between elongator tRNAs in the genome, and is scored “one.” Repeating the comparison for all seventy three positions (i.e., the number of bases in the tRNA molecule), and then for the number of species being compared (in this example, eighteen genomes), yields the global frequency of discriminatory positions. Because eighteen genomes have been examined, the maximum base discrimination frequency is 18 (denoting perfect dissimilarity), and the minimum value is 0 (denoting perfect identity).

The methods of the present invention thus provide a means by which a number of components (for example, nucleic acid sequences, amino acid sequences, carbohydrate chains, and the like) can be compared to one another across species, and differences which are conserved across species highlighted.

Interactions with Cellular Components

In a further step, the positions that were determined to have the greatest sum value can be assessed for their ability to interact with a cellular factor, such as a protein, a peptide, a protein complex, a nucleic acid, a protein-nucleic acid complex, a carbohydrate chain, or a combination of these factors. As one example, the position(s) identified by the methods of the present invention may interact with an enzyme at, for example, an active site or a regulatory site. As another example, the identified position(s) may interact with a protein-nucleic acid complex, e.g., a ribosome.

Interactions with cellular components can be determined by a number of techniques known to those in the art. Optional assays include radiolabel assays, FACS-based assays, agglutination assays, antibody binding assays, NMR spectroscopy binding analyses, and the like. Alternatively, molecular modeling studies can be performed to examine interactions between components, using software available publicly (see, for example, the NIH Center for Molecular Modeling, www.cmm.info.nih.gov/modeling/gateway.html) or commercially (from, e.g., Hypercube Inc., Gainesville Fla.; MDL Information Systems, San Leandro, Calif.; Molecular Applications Group, Palo Alto, Calif.; Molecular Simulations, Inc, San Diego, Calif.; Oxford Molecular Group PLC, London, UK; and Tripos, Inc., St. Louis, Mo.).

Modified Elements

In addition to the steps described above, the methods of the present invention can further include the step of determining whether the identified positions contain modified elements, for example, amino acids, nucleotides, or carbohydrate elements that have been methylated, alkylated, acetylated, esterified, ubiquitinated, lysinylated, sulfated, phosphorylated, glycosylated, and the like.

In embodiments of the present invention in which the set of similar sequence strings are tRNA sequences, the modified element can be a modified nucleic acid element. Known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington DC). Exemplary modified RNA elements include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyluridine; 5-carboxymethylaminomethyl-2thiouridine; dihydrouridine; dihydroribosylthymine; 2′-0-methyladenosine; 2-methyladenosine; N⁶N-methyladenosine; N⁶, N⁶-dimethyladenosine; N⁶,2′-O-trimethyladenosine; 2-methylthio-N⁶N⁶-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N6-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′-O-methyl inosine; 1-methyl inosine; 1;2′-O-dimethyl inosine; 2′-O-methyl guanosine; 1-methyl guanosine; N²-methyl guanosine; N²,N²-dimethyl guanosine; N², 2′-O-dimethyl guanosine; N², N², 2′-O-trimethyl guanosine; 2′-O-ribosyl guanosine (phosphate); 7-methyl guanine; N2;7-dimethyl guanosine; N²; N^{2;7}-trimethyl guanosine; wyosine; methylwyosine; undermodified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine. The methods of the present invention can identify additional modified nucleic acid elements.

In embodiments of the present invention in which the set of similar sequence strings are amino acid sequences, the modified element can be a modified amino acid element. Common modifications to amino acids include phosphorylation of tyrosine, serine, and threonine residues; methylation of lysine residue; acetylation of lysine residues; hydroxylation of proline and lysine residues; carboxylation of glutamic acid residues; and glycosylation of serine, threonine, or asparagine residues. Other modifications include, but are not limited to, attachment of a ubiquitin molecule (a 76-amino acid polypeptide involved in targeting of protein degradation) to lysine residues. The methods of the present invention can identify additional modified amino acid elements.

In embodiments of the present invention in which the set of similar sequence strings are carbohydrate sequences, the modified element can be a modified carbohydrate element or modified sugar. Common modifications to carbohydrate sugars include, but are not limited to, addition of sulfates, phosphates, amino groups, carboxyl groups, sialyl groups, additional sugar residues, and the like. The methods of the present invention can be used to identify additional modified sugar or carbohydrate elements.

Determination of whether the similar sequence strings contain modified elements involves the preparation of assay solutions containing the similar sequence strings and analysis of the contents. Optionally, the similar sequence strings can be isolated and/or purified during the preparation of the assay solution. The technique(s) used in the isolation of the similar sequence strings will depend upon the type of sequence string involved; methods for the isolation and/or purification of sequence strings such as peptides and proteins, nucleic acids, and carbohydrates are known in the art, and include, but are not limited to, the following techniques: size exclusion chromatography, affinity chromatography, gel filtration, high pressure liquid chromatography (BIPLC), isoelectric focusing, multi-dimensional electrophoresis techniques, salt precipitation, density-gradient centrifugation, and the like.

Methods and techniques for compound analysis are also well known in the art. Some preferred analytical techniques for use in determining whether an element of a similar sequence string has been modified, the extent of modification, and/or the type of modification include, but are not limited to, mass spectrometry, thin layer chromatography (TLC), HPLC, capillary electrophoresis (CE), NMR spectroscopy, X-ray crystallography, cryo-electron microscopic analysis, or a combination thereof.

Mass spectrometry is a particularly versatile analytical tool, and includes techniques and/or instrumentation such as electron ionization, fast atom/ion bombardment, MALDI (matrix-assisted laser desorption/ionization), electrospray ionization, tandem mass spectrometry, and the like. A brief review of mass spectrometry techniques commonly used in biotechnology can be found, for example, in Mass Spectrometry for Biotechnology by G. Siuzdak (1996, Academic Press, San Diego).

In the methods of the present invention, the assay solutions (containing the similar sequence strings) are prepared for mass spectrometry by preparing the sequence strings in a suitable solvent system. Suitable solvent systems include, but are not limited to H₂O, methanol, CHCl₃, CH₂Cl₂, DMSO (dimethyl sulfoxide), THF (tetrahydrofuran) and TFA (trifluoroacetic acid). Optionally, the sample can be desalted prior to analysis.

Alternatively, the assay solutions containing the similar sequence strings are prepared for NMR spectroscopy by removal of the original solvent solution (for example, by lyophilization), and re-dissolution into a stable-isotope solvent, such as a deuterated solvent. Suitable deuterated solvents include, but are not limited to D₂O (deuterium oxide), CDCl₃, DMSO-d6, acetone-d6, and the like (available, for example, from Cambridge Isotope Labs, Andover, Mass.; www.isotope.com). Optionally, the samples can be analyzed using LC-NMR spectroscopy. Analysis by these methodologies can provide information related to both the presence of one or more modifications, as well as the type or identity of the modification (see, for example, NMR of Macromolecules: A Practical Approach, G. C. K. Roberts, ed., 1993, Oxford University Press, New York).

Computers and Logical Instructions

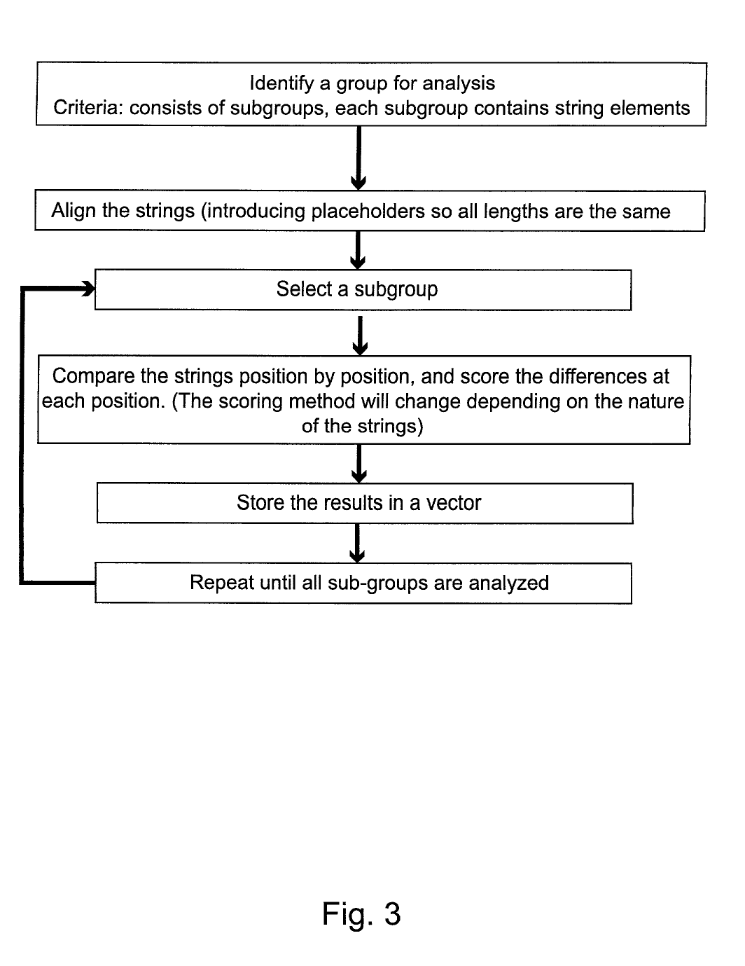

The present invention also provides a computer or computer readable medium having one or more logical instructions for identifying at least one conserved difference in a set of similar sequence strings derived from a plurality of species. One embodiment of the computer or computer-readable medium of the present invention is depicted in FIG. 3. Typically computer 100 includes central processing unit (CPU) 107 and monitor 105. Optionally, CPU 107 comprises a hard drive, and computer 100 includes one or more additional drives 115 (such as a floppy drive, a CD-ROM, etc.) The computer or computer-readable medium can also include one or more user interfaces, such as keyboard 109 and/or mouse 111, and thus can be accessed by a user.

Optionally, the computer or computer-readable medium further comprises database 120 comprising one or more sets of sequence strings. The one or more sets of sequence strings can be obtained from a number of sources, including, but limited to public and/or private databases. In one embodiment of the computer of the present invention, database 120 is in communication with hard drive 107 via communication medium 119. Thus, database 120 need not be located proximal to CPU 107.

The computer or computer readable medium can be operated using any available operating system (commercial or otherwise), or it can be another form of computational device known to one of skill in the art.

The computer or computer readable medium can use logical instructions to compare at least n sequence elements in a first similar sequence string to at least n sequence elements in a second similar sequence string, for a first species of the plurality of species. The logical instructions assign a value to each of n positions of the at least n sequence elements, based upon whether the sequence elements are identical or different in the two similar sequence strings. The comparing and assigning process is repeated by the logical instructions for each species in the plurality of species. The values assigned for each of the n positions are added together for each position across the plurality of species. The positions having the greatest sum value are determined, thus identifying the positions of conserved difference in the set of similar sequence strings.

Logical instructions for performing the above-described calculations can be constructed by one of skill using a standard programming language such as C, C++, Visual Basic, Fortran, Basic, Java, or the like. For example, a computer system can include software for analyzing one or more sets of similar sequence strings, and optionally modified for communication with a user interface (e.g., a GUI in a standard operating system such as a Windows, Macintosh, UNIX, LINUX, and the like), to obtain the sequence strings, align the component elements, perform the calculations, and/or manipulate the examination results (i.e. the identified positions of conserved differences). Standard desktop applications including, but not limited to, word processing software (e.g., Microsoft Word™ or Corel WordPerfect™), spreadsheet and/or database software (e.g., Microsoft Excel™, Corel Quattro PrOT Microsoft Access™, Paradox™, Filemaker Pro™, Oracle™, Sybase™, and Informix™) and the like, can be adapted for these (and other) purposes.

Optionally, the computer or computer readable medium can provide the examination results in the form of an output file. The output file can, for example, be in the form of a graphical representation of part or all of the sets of similar sequence strings.

In another embodiment of the present invention, the computer or computer readable medium can further comprise logical instructions for providing the sets of similar sequence strings. The sets of similar sequence strings can be derived, for example, from longer sequences (for example, from genomic sequences in the case of nucleic acid sequences, or from pro-forms of proteins in the case of amino acid sequences). Sets of similar sequence strings can be obtained, for example, by using such logical instructions (e.g., a computer-based searching algorithm) to analyze larger sequences or collections of sequences, and identify the desired target sequences. One example of logical instructions for providing sets of similar sequence strings that can be used in the present invention is “tRNAscan-SE,” tRNA analysis software available from Washington University in St. Louis (http://www.genetics.wustl.edu/eddy/tRNAscan-SE/). The tRNAscan-SE program is distributed as open software under the terms of the GNU License (see http://www.gnu.or/copyleft/gpl.html for further information).

Uses of the Methods, Devices and Compositions of the Present Invention

Modifications can be made to the method and materials as described above without departing from the spirit or scope of the invention as claimed, and the invention can be put to a number of different uses, including:

The use of any method herein, to identify any composition or collection of positions of conserved differences within a set of similar sequence strings.

The use of a method or an integrated system to identify one or more positions of conserved differences within a set of similar sequence strings.

An assay, kit or system utilizing a use of any one of the selection strategies, materials, components, methods or substrates hereinbefore described. Kits will optionally additionally comprise instructions for performing methods or assays, packaging materials, one or more containers which contain assay, device or system components, or the like.

In an additional aspect, the present invention provides kits embodying the methods and devices herein. Kits of the invention optionally comprise one or more of the following: (1) a set of similar sequence strings as described herein; (2) one or more logical instructions for providing and/or analyzing the set of similar sequence strings; (3) a computer or computer-readable medium for performing the methods of the present invention and/or for storing the examination results; (4) instructions for practicing the methods described herein; and, optionally, (5) packaging materials.

In a further aspect, the present invention provides for the use of any component or kit herein, for the practice of any method or assay herein, and/or for the use of any apparatus or kit to practice any assay or method herein.

### EXAMPLE 1

Analytical Procedure for Determining Sites of Conserved Differences

The sites of conserved differences, or dissimilarity, can be determined using matrix theory. One embodiment of this approach is as follows:

1. Define set G={g₁, g₂, . . . gₙ}

2. Define subset gᵢ={s₁, s₂}, where S_{I} is a string of length j and s₂ is a string of length k, k≧j .

3. Define R, the alignment of all strings in subsets {g₁, g₂, . . . gₙ}. The aligned strings are in some cases lengthened by the insertion of placeholders so that, after alignment, all strings in G have the same number of characters, l. The subsets of these length-equalized strings are designated as for example subset γᵢ={σ₁, σ₂}. The collection of all γᵢ comprise Γ.

4. For each subset of Γ, γᵢ, define a matrix, Aᵢ, dimension 2×l. Row 1 of Aᵢ contains the 1 to lth character of string σ₁,an element of subset γᵢ and row 2 of A_{I} contains the 1 to lth characters of string σ₂. Each column of Aᵢ therefore contains a pair of aligned elements from corresponding positions of the strings, σ₁, σ₂, that comprise set γᵢ.

5. Define matrix D, dimension 1×l. Populate matrix D with zeros. For each subset γᵢ, i=1 to n:

a) Create matrix Aᵢ

b) Populate: A_{l,i} with characters from strings σ₁, and A_{1,i} with characters from string σ₂.

c) For each column c of Aᵢ 1 to l, if position (1,c) of Aᵢ=(2,c) of Aᵢ, let D_{c}=D_{c}+0; else let D_{c}=D_{c}+1.

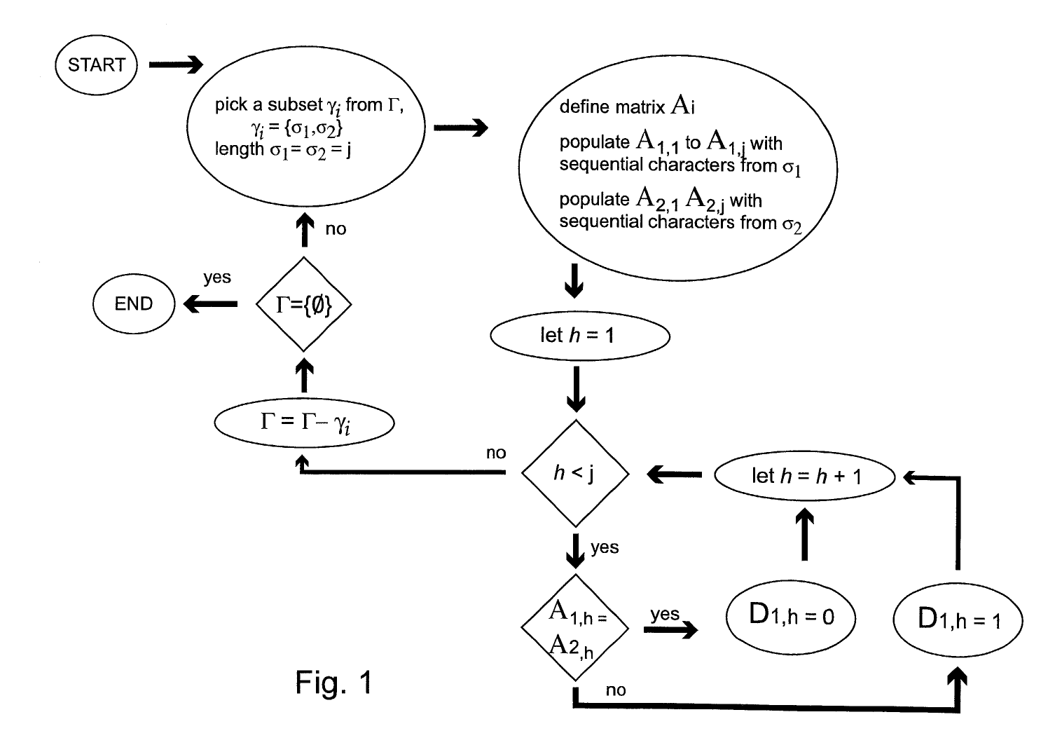

This embodiment of the present invention is depicted in schematic form in FIG. 1. The address of the largest value stored in D_{c} is the position most frequently dissimilar between the string pairs of each sub-set γᵢ.

### EXAMPLE 2

Alternate Procedure for Determining Sites of Conserved Differences

An alternate embodiment of the modeling involved in determining sites of conserved difference in sets of sequence strings is described as follows:

Define set G={g₁, g₂, . . . gₙ}. Set G comprises a plurality of species and can be any collection of n items, such as species of bacteria, make and model of cars, etc. Each member, or species, of set G is represented by subset gₓ={Sⱼ, Sₖ}, where sⱼ is a sequence string of length j and sₖ is a string of length k. The sequence strings sⱼ and sₖ are comprised of the component elements to subsequently be compared for conserved regions of difference. Optionally, each species contributes at least two similar sequence strings; thus, in the present example, subset gₓ is comprised of two sequence strings sⱼ and Sₖ. Alternatively, some or all of the species in set G can contribute multiple (i.e., more than two) similar sequence strings.

Having established set G and subsets g₁, g₂, . . . gₙ, the component sequence strings of the n subsets are then aligned prior to comparison. In some cases, alignment is achieved by the insertion of placeholder elements so that, after alignment, all of the sequence strings originally present in G have the same number of elements, L. Elements can, for example, be added to one or more positions, including the beginning, the end, or within the sequence string, in order to align the sequences for analysis. Set H (comprising h₁, h₂, . . . hₙ) represents the aligned subsets of G.

Matrix (A) is defined having n rows and L columns. To populate the positions in row i of matrix A, the elements at the corresponding positions of subset hᵢ are examined. If the sequence elements are identical, a “zero” is placed in that position of the matrix. If the sequence elements are dissimilar, then a value representing the number of events of dissimilarity is placed in the matrix position. For analysis of a sib-pair, this value would be “one” if the element at position I was different (i.e. one instance of dissimilarity). For example, if aligned subset h3 has the same element at position 5 in both s1 and s2, then matrix A has a “zero” at row 3, column 5 (i.e., A[3,5]=0). And if aligned subset h3 has differing elements at position 6 in both s1 and s2, then matrix A has a “one” at row 3, column 6 (i.e., A(3,6)=1). This comparison is repeated for each of the L positions of each of the n subsets of sequence strings to fully populate the matrix.

Finally, the values in the L columns of matrix A are added together. The position, or “address” of the largest value in matrix A corresponds to the position most frequently dissimilar between the string pairs of collection G.

### EXAMPLE 3

Analysis of tRNA Sequences from Bacteria

The tRNA genes from genomic DNA sequences of eighteen bacterial species were examined for one or more positions of conserved differences. The plurality of species included a wide sampling of prokaryotic life forms, including Eubacteria and Archaea. Sets of similar tRNA sequences were derived from a number of species, including obligate intra-cellular parasites (Chlamydia trachomatis, Chlamydia pneumoniae, Ricketsia prowesekii, and Mycobacterium tuberculosis); obligate extra-cellular parasites (Mycoplasma genitalium and Mycoplasma pneumoniae); four distantly related opportunistic human pathogens (Treponema pallidum, Borrelia burgdorferi, Helicobacterpylori, Haemophilus influenzae); a ubiquitous enteric comensal (Escherichia coli); an industrially important gram positive bacterium (Bacillus subtilis), a methanogen (Methanococcus jannaschii), a cyanobacterium (Synechocystis sp.); and a number of extremophiles (Archaeoglobus fulgidus, Methanobacterium thermatrophicum, Pyrococcus horikoshuii, and Aquifex aeolicus). Because the plurality of species included representatives of a variety of divergent bacterial species, generalizations which emerge from comparative analysis of the set can be applied to most bacterial species, including those not present in the sample. Certain trends occur without exception in this sample and may be universal among prokaryotes.

Similar sequence strings of tRNA genes were obtained from the complete DNA sequences of the eighteen bacterial genomes as follows. Genomic DNA sequences are available from public sources via the internet; the selected genomic sequences were downloaded to a computer for comparison and analysis (see Table 2 for Internet addresses used as sources of sequence information for each species). In addition, tRNA analysis software (tRNAscan-SE) was acquired from the Washington University, St. Louis (http://www.genetics.wustl.edu/eddy/tRNAscan-SE/). The nucleic acid sequence of each genome was searched for tRNA sequences using the tRNAscan-SE program, setting the program parameters to the most comprehensive values (i.e., with the lowest probability of missing a tRNA gene). The resulting sets of similar sequence strings were then examined to identify one or more positions of conserved differences among species.

TABLE 2
INTERNET ADDRESSES OF BACTERIAL GENOME PROJECTS
AND ABBREVIATIONS FOR EACH BACTERIAL SPECIES

| Bacterium | abbrev. | Web address |
|---|---|---|
| Haemophilus | Hi | http://www.tigr.org/tdb/mdb/mdb.html |
| influenzae | | |
| Mycoplasm | Mg | http://www.tigr.org/tdb/mdb/mdb.html |
| genitalium | | |
| Helicobacter | Hp | http://www.tigr.org/tdb/mdb/mdb.htm1 |
| pylori | | |
| Archaeoglobus | Af | http://www.tigr.org/tdb/mdb/mdb.html |
| fulgidus | | |
| Borrelia | Bb | http://www.tigr.org/tdb/mdb/mdb.html |
| burgdorferi | | |
| Treponema | Tp | http://www.tigr.org/tdb/mdb/mdb.html |
| pallidum | | |
| Methanococcus | Mj | http://www.tigr.org/tdb/mdb/mdb.html |
| jannaschii | | |
| Rickettsia | Rp | http://evolution.bmc.uu.se/˜siv/gnomics/Rickett |
| prowazekii | | sia.html |
| Escherichia | Ec | http://www.genetics.wisc.edu:80/index.html |
| coli | | |
| Bacillus | Bs | http://www.pasteur.fr/recherche/SubtiList.html |
| subtilis | | |
| Chlamydia | Ct | http://chlamydia-www.berkeley.edu:4231/ |
| trachomatis | | |
| Chlamydia | Cp | http://chlamydia-www.berkeley.edu:4231/ |
| pneumoniae | | |
| Mycoplasma | MP | http://www.zmbh.uni- |
| pneumoniae | | heidelberg.de/M_pneumoniae/MP_Home.html |
| Aquifex | Aa | http://www.biocat.com/ |
| aeolicus | | |
| Methano- | Mt | http://www.genomecorp.com/genesequences/m |
| bacterium | | ethanobacter/abstract.html |
| thermoauto- | | |
| trophicum | | |
| Synechocystis | Sy | http://www.kazusa.or.jp/cyano/cyano.html |
| sp. | | |
| Mycobacterium | Mt | http://www.sanger.ac.uk/Projects/M_tubercul |
| tuberculosis | | osis/ |
| Pyrococcus | Ph | http://www.bio.nite.go.jp/ot3db_index.html/ |
| horikoshii | | |

Bacterial tRNA Genes

The comprehensive survey performed using the methods and devices of the present invention revealed several unexpected findings, including the observations that 1) none of the bacterial species examined possessed a separate tRNA gene for each of the sixty-one amino-acid specifying codons, which suggests that one or more of the encoded tRNAs must either be “multi-functional” or exist in multiple (i.e. modified) states having separate specificities, 2) there is a prominent and strongly conserved preference for particular anticodons in tRNA sets, and 3) some potential anticodons are completely censored (i.e., the anticodon does not occur in the plurality of genomes examined). This information can be used for directing pharmaceutical research towards more specific (or, conversely, nonspecific) drug targets. For example, the methods and devices of the present invention reveal that the unusual amino acid selenocysteine is selectively utilized in only five of the eighteen species analyzed, suggesting that the biosynthetic machinery involved in selenocysteine biosynthesis and/or utilization could be targeted in a species-specific manner.

TABLE 3
TOTAL tRNA GENE TYPES VERSUS TOTAL NUMBER
OF tRNA GENES

| | Number of | Number of |
|---|---|---|
| Bacterial Species | tRNA gene types | tRNA genes |
| Mycoplasma genitalium | 34* | 37* |
| Mycoplasma pneumoniae | 34* | 38* |
| Chlamydia trachomatis | 35 | 37 |
| Rickettsia prowesekii | 30 | 33 |
| Treponema pallidum | 42 | 44 |
| Chlamydia pneumoniae | 36 | 38 |
| Borellia burgdorferi | 29 | 31 |
| Aquifex aeolicus | 39* | 43* |
| Helicobacter pylori | 33 | 36 |
| Methanococcus jannaschii | 33* | 37* |
| Methanobacterium | 33 | 37 |
| thermoautotrophicum | | |
| Pyrococcus horikoshii | 42 | 44 |
| Heamophillus influenzae | 32 | 51 |
| Archaeoglobus fulgidus | 43 | 46 |
| Synechocystis sp. | 39 | 41 |
| Bacillus subtilis | 34 | 84 |
| Mycobactenium | 43 | 45 |
| tuberculosis | | |
| Escherichia coli | 40* | 87* |

*Includes one seleno-cysteine tRNA

Frequency of Bases in the Anticodon “Wobble” Position

Interactions between the three bases in a given codon of a MRNA sequence and the matching bases in the anticodon region of a tRNA molecule take place via base-pairing. However, the third position in the codon:anticodon pair (i.e. the third base in the codon, and the first base in the anticodon) does not always follow the usual base-pairing rules, because the conformation of the anticodon loop allows some flexibility at this position during the codon:anticodon interaction. Thus, this position, termed the “wobble” position, is not limited to a single base pair interaction. However, this loss of uniqueness to the third determinant position in a given codon is often irrelevant in determining the amino acid to be added to the nascent peptide chain, due to a coevolved degeneracy in the genetic code. (For a review of the wobble hypothesis, see, for example, Chapter 9, “Interpreting the Genetic Code” by Lewin (1997), Genes VI, Oxford University Press, Oxford, UK.).

Sixteen of the sixty four theoretical tRNA types (as defined by their anticodon sequences) have an adenosine base (a) at position 34, the “wobble position” of the anticodon. Using the methods of the present invention, it was determined that twelve of the sixteen potential “a—” anticodons were not found in any of the bacterial genomes examined (i.e., they are “censored” anticodons). The censored anticodons beginning with ‘a’ were aaa, aua, aag, aug, aau, agu, auu, acu, aac, agc, auc, and acc. Three of the remaining four wobble adenosine anticodons (aga, aca, and agg) were “under-represented,” since they occur in less than 50% of the genomes analyzed. The anticodon “acg” occurred in eleven of the eighteen genomes.

Likewise, sixteen tRNA types have a cytosine base (c) at the wobble position of the anticodon. It is interesting to note that seven of the “c—” tRNA types were underrepresented (cgg, cug, cuu, cac, cgc, cuc, ccc). However, none of the tRNA types having a cytosine in the wobble position of the anticodon were censored.

A single anticodon with a wobble uridine (u), the anticodon “uau,” is censored in the eighteen bacterial genomes. None of the remaining fifteen wobble uridine anticodons are under-represented.

No anticodon containing a guanosine (g) at the wobble position is censored, nor is any member of this anticodon subset underrepresented.

Analysis of Methionyl tRNA Genes

The anticodon cau defines the methionyl transfer RNA. This gene occurs three or more times in each of the eighteen genomes examined. This is the only tRNA type which occurs multiple times in all bacterial genomes. Methionine is the first amino acid in most bacterial proteins, and there is a special ‘initiator’ tRNA which is used to initiate protein synthesis from each gene, while the “elongator” tRNA-met contributes methionine residues within the growing peptide chain.

Three structural features characterize the methionyl initiator tRNA molecule: unpaired bases at the top of the acceptor stem, a conserved a::u base pair in the D-stem between position 11 and position 24, and a stack of two to three g::c base pairs in the anticodon stem. Using these features it is possible to sort the methionyl tRNAs from each genome into subsets, and to count the number of initiator methionyl tRNAs in each genome. The number of initiator and elongator methionyl tRNA genes is presented in Table 4. In sixteen of the eighteen genomes there are three methionyl tRNA genes; in these triplicate sets there is always one initiator methionyl tRNA and two elongator methionyl tRNA genes. B. subtilis has a total of five methionyl tRNA genes, two of which are initiator genes. E. coli has eight methionyl tRNA genes, four of which are initiators.

TABLE 4
BREAKDOWN OF METHIONYL tRNA GENE SETS BY
INITIATOR/ELONGATOR SUBTYPES

| | Total | | |
|---|---|---|---|
| | Number | Number of | Number of |
| | tRNA-Met | Initiator | Elongator |
| Bacterial Species | Genes | tRNA-Met | tRNA-Met |
| Mycoplasma genitalium | 3 | 1 | 2 |
| Mycoplasma pneumoniae | 3 | 1 | 2 |
| Chlamydia trachomatis | 3 | 1 | 2 |
| Rickettsia prowesekii | 3 | 1 | 2 |
| Treponema pallidum | 3 | 1 | 2 |
| Chlamydia pneumoniae | 3 | 1 | 2 |
| Borellia burgdorferi | 3 | 1 | 2 |
| Aquifex aeolicus | 3 | 1 | 2 |
| Helicobacter pylori | 3 | 1 | 2 |
| Methanococcus jannaschii | 3 | 1 | 2 |
| Methanobacterium | 3 | 1 | 2 |
| thermoautotrophicum | | | |
| Pyrococcus horikoshii | 3 | 1 | 2 |
| Heamophillus influenzae | 3 | 1 | 2 |
| Archaeoglobus fulgidus | 3 | 1 | 2 |
| Synechocystis sp. | 2 | 0 | 2 |
| Bacillus subtilis | 5 | 2 | 3 |
| Mycobacterium tuberculosis | 3 | 1 | 2 |
| Escherichia coli | 8 | 2 | 6 |

Analysis of Elongator tRNA-Met Genes

Sets of similar sequence strings comprising elongator methionyl tRNA (tRNA-Met) gene sequences were analyzed for positions of conserved difference, using the methods of the present invention. The differences among elongator tRNA-Met subtypes were systematically identified by the process of disjunction analysis as described above. Using this statistical process, the elements in sets of paired elongator methionyl tRNA sequences were examined for variations between the sib-pairs. Such variations suggest functionally important features.

For each pair of elongator tRNA-Met genes, the sequences were aligned and the component elements were compared, base for base, starting at position one and proceeding through the tRNA to position seventy-three. Positions having non-identical elements were assigned a value of one, while positions having identical elements were assigned a value of zero. For example, in Bacterium sp., if elongator tRNA-1 is compared to elongator tRNA-2, and at position 2 the base ‘g’ occurs in elongator tRNA-1 and the same base, a ‘g’ occurs in elongator tRNA-2, then the position 2 is scored ‘zero’ in that genome. At position three, tRNA-1 might be ‘a’, while tRNA-2 might be ‘g’. This is a ‘discriminatory position’ between elongator tRNAs in the genome, and is scored ‘one’. Repeating the comparison for all positions, and then for all genomes, yields the global frequency of discriminatory positions. Because 18 genomes have been examined the maximum base discrimination frequency is 18 (denoting perfect dissimilarity), and the minimum value is 0 (denoting perfect identity).

In sixteen of the bacterial genomes examined, there were two elongator tRNA-Met genes. The tRNAs in these subsets are not identical genes. In two of the bacterial genomes there were more than two elongator methionyl tRNA genes. B. subtilis has three such genes, and E. coli has four. In these two cases the additional elongator tRNAs are duplicates of members of the two “basic” elongator tRNA-Met gene subsets, and can be grouped by sequence identity. In other words, each of the eighteen bacterial genomes has two different elongator tRNA-Met subtypes to be analyzed.

The distribution of the identified points of conserved base differences between members of the two elongator tRNA subsets is not random. These “discrimination positions” occur in two clusters, one around position five, and one around position forty-four, of the tRNA sequence. Position five is a discriminatory base in sixteen of the eighteen genomes (i.e., in all the bacterial species examined except Chlamydia trachomatis and Chlamydia pneumoniae). Position forty-four is discriminatory in all eighteen genomes. The identification of discriminatory position 44 in all eighteen elongator methionyl tRNA sib pairs implies that all sib pairs are under selection by a similar molecular interaction at position 44 such as recognition of one sib from each pair by an enzyme. The present invention also provides compounds which interact at one or more of these discriminatory positions.

Modified Elements: Lysidine

Lysinylation is the biochemical modification of cytidine by the addition of lysine to position 2 of the cytidine base. The resulting hyper-modified base is called lysidine. The reaction is known to occur post-transcriptionally on the cytosine found at position 34 (i.e., within the anticodon region) of a particular “methionyl” tRNA in E. coli, B. subtilis, and M. caprolicum. Conversion of the tRNA-Met position 34 cytosine to lysidine imposes a complete functional transformation of the tRNA. Unmodified, the tRNA-Met associates with the methionyl codon AUG, as would be expected based on its native anticodon sequence (cau). The unmodified tRNA-Met is recognized by the appropriate aminoacyl tRNA synthetase and is correctly charged with methionine. However, upon lysinylation of the cysteine in position 34, the modified tRNA-Met* recognizes a different codon, the triplet AUA (an isoleucine codon), and no longer reads the methionyl codon AUG. Furthermore, lysinylation strongly inhibits interaction of the modified tRNA-Met* with methionyl tRNA synthetase. Thus the lysinylated tRNA-Met* is charged with the amino acid isoleucine, coupling the isoleucyl codon AUA to its proper amino acid through the modified (lysinylated) tRNA.

Two distinct elongator methionyl tRNAs are found in all bacteria examined. The methods of the present invention were used to analyze the tRNA-Met sequence strings from these species and determine whether the sib-pairs possessed discriminator bases that allow each sib to be distinguished from its mate. These features form a molecular basis for recognition of the appropriate elongator “methionyl” tRNA by the lysinylation enzyme(s).

Analysis of Selenocysteine tRNA Genes

Another observation based upon the methods of the present invention concerns the occurrence of tRNA types which read selenocysteine. Often, the selenocysteine residue plays a role in the catalytic activity of the protein (for example, redox reactions). In five of the bacterial genomes examined, the codon TGA, which is normally utilized as a translation stop codon, appears to encode the rare amino acid selenocysteine. These species, Mycoplasma genitalium, M. pneumoniae, Aquifex aeolicus, Methanococcus jannaschii, and Escherichia coli, have predicted tRNA genes with the complementary anticodon, uca. These five species are equipped to incorporate selenocysteine into proteins.

### EXAMPLE 4

Determination and Analysis of Positive or Negative Selection Among Alleles in a Population

Methods in which higher order analyses are performed can be used in a number of applications, for example, to analyze a population of sister chromatids to detect positive or negative selection for heterozygosity on a polymorphic allele.

Under the rules of Mendelian segregation, a bimorphic allele (such as A and A′) will segregate to produce three genotypes: two homozygous classes (A/A and A′/A′) and one heterozygous class (A/A′). Under a purely stochastic regimen heterozygotes will reach an equilibrium frequency in the population of 50%. Deviation from 25:25:50 frequency is prima facia evidence of non stochastic assortment. Comparable, or “balanced” A/A and A′/A′ frequencies together with a statistically-relevant deviation from 50% for the heterozygote indicates negative(<50% A/A′) or positive (>50% A/A′) selection for the heterozygotic state.

Polymorphic alleles will segregate to form multiple genotypes. For example, a trimorphic allele (such as A, A′, and A″) will segregate into six genotypes, three homozygous genotypes (AA, A′A′ and A″A″) and three heterozygous genotypes (AA′, AA″, and A′A″). A “quatro”-morphic allele (A, A′, A″, A′″) will segregate into ten genotypes, four homozygous (AA, A′A′, A″A″, and A′″A′″) and six heterozygous genotypes, and so forth. Higher order analyses of the dispersion of the alleles can be used to analyze associated traits and frequency of retention.

A well known example of positive selection on heterozygosity is the so-called sickle cell allele Hs of β-hemoglobin (having a glutamic acid→valine substitution at position six). The homozygous “sickled” genotype Hs/Hs is highly deleterious. However, H/Hs heterozygosity confers resistance to infection by Plasmodium falciparum; the lack of resistance leads to malaria and is often fatal. H/Hs heterozygotes are therefore more frequent in the population than expected for a lethal homozygous recessive allele.

The methods of the present invention can be employed to detect positive, negative or neutral selective environments for any polymorphic allele. The principle is illustrated for the case of a bimorphic allele A, A′. The predicted frequencies for n-morphic alleles (n>2), generalize in the obvious way under well known combinatorial rules.

The complete DNA sequence of human chromosomes can be obtained by a variety of methods. Shotgun sequencing is one such method. Since DNA is purified in bulk prior to the sequencing process, sequence from both sister chromatids is obtained. In general, the sequence is identical on both chromatids. The exception is at polymorphic loci. for example, bimorphic loci. For any pair of sister chromatids, at a heterozygous site about half of the sequences will report state A and half of the sequences state A′. The methods of the present invention can be used to identify these sites on conserved differences. However, not all pairs of sister chromatids will be polymorphic at a particular site. Many will display A/A or A′/A′, which the algorithm reports as similarities. The frequency of dissimilar pairs A/A′ in the total population will equal<<50%, ˜50%, or >>50%.

### EXAMPLE 5

Higher Order Comparisons of Regions of Dissimilarity

The previous examples depict a simple, pair-wise comparison between “sibling” sequence strings (subsets of two) within a larger set. In that embodiment of the methods of the present invention, each character in each pair of sequence strings assumes one of two states (e.g., on/off, true/false, 0/1). Another embodiment can be envisioned in which the subsets contain more than two “sibling” sequence strings. The methods of the present invention can be applied to fields (and sets of items) outside of the area of bioinformatics.

As an example, consider the superset of Masonic Lodges in California. The membership of each lodge constitutes a subset of two or more individuals. A survey might be devised so that all questions must be answered “yes” or “no”. Such yes/no responses can then be encoded as 1/0 and each individual in each subset can be represented as a bit string that encodes the responses to the survey. Then, within each subset, each bit-string can be entered as a row in a matrix. Summing down each column then dividing by the number of rows gives the relative frequency. These scores can be collected in a scoring matrix and an average frequency at each position in the bit string calculated for all subsets, An average frequency score close to 0.5 indicates maximum dissimilarity for responses to the survey for the corresponding question.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for identifying one or more positions of conserved difference in a set of similar sequence strings, the method comprising:
providing a set of similar sequence strings derived from a plurality of species, wherein each similar sequence string comprises at least n sequence elements;
comparing the at least n sequence elements in a first similar sequence string to the at least n sequence elements in a second similar sequence string, for a first species of the plurality of species;
assigning a value to each of n positions of the at least n sequence elements, based upon whether the sequence elements are identical or different in the two similar sequence strings;
repeating the comparing and assigning for each species in the plurality of species;
summing the values assigned for each of the n positions across the plurality of species; and
identifying which of the n positions have the greatest sum value, thereby identifying the positions of conserved difference in the set of similar sequence strings.

2. The method of claim 1, wherein each species in the plurality of species contributes at least two similar sequence strings to the set of similar sequence strings.

3. The method of claim 1, wherein each species in the plurality of species contributes more than two similar sequence strings to the set of similar sequence strings.

4. The method of claim 1, wherein the providing a set of similar sequence strings comprises:
providing a set of sequences;
providing logical instructions for recognizing a target sequence string; and
using the logical instructions to analyze the sequences and identify the target sequence strings, thereby providing a set of similar sequence strings.

5. The method of claim 1, wherein the set of similar sequence strings comprises sets of amino acid sequences, nucleic acid sequences, lipid-based sequences or carbohydrate sequences.

6. The method of claim 5, wherein the set of similar sequence strings comprises a set of tRNA molecules.

7. The method of claim 5, wherein the set of similar sequence strings comprises a set of alleles.

8. The method of claim 7, wherein the set of alleles comprises at least two alleles.

9. The method of claim 7, wherein the set of alleles comprises more than two alleles.

10. The method of claim 1, wherein the plurality of species comprises a plurality of prokaryotic species, eukaryote species, or combinations thereof.

11. The method of claim 8, wherein the plurality of prokaryotic species comprises a plurality of eubacteria species, archaea species, or combinations thereof.

12. The method of claim 1, wherein the comparing and assigning is performed in a computer.

13. The method of claim 1, further comprising determining whether the positions that have the greatest sum values comprise elements which interact with a protein, a peptide, a protein complex, a nucleic acid, a protein-nucleic acid complex, a carbohydrate chain, or a combination thereof.

14. The method of claim 13, wherein the protein comprises an enzyme.

15. The method of claim 13, wherein the protein-nucleic acid complex comprises a ribosome.

16. The method of claim 1, further comprising determining whether the positions that have the greatest sum values comprise modified elements.

17. The method of claim 16, wherein the modified elements comprise amino acids or nucleotides which are modified by methylation, acetylation, ubiquitination, lysinylation or glycosylation.

18. A method for identifying one or more positions of conserved difference in a set of similar sequence strings, the method comprising:
providing a set of similar sequence strings derived from a plurality of species, wherein each similar sequence string comprises at least n sequence elements, and wherein each species in the plurality of species contributes two or more similar sequence strings to the set of similar sequence strings;
simultaneously comparing the at least n sequence elements for the two or more similar sequence strings from a first species of the plurality of species;
assigning a value to each of n positions of the at least n sequence elements, based upon whether the sequence elements are identical or different in the two or more similar sequence strings;
repeating the comparing and assigning for each species in the plurality of species;
summing the values assigned for each of the n positions across the plurality of species; and
identifying which of the n positions have the greatest sum value, thereby identifying the positions of conserved difference in the set of similar sequence strings.

19. The set of conserved differences in a set of similar sequence strings as identified by the method of claim 1.

20. A computer or computer-readable medium comprising one or more logical instructions for identifying at least one conserved difference in a set of similar sequence strings derived from a plurality of species,
wherein each species in the plurality of species comprises at least two similar sequence strings; and
wherein the logical instructions compare at least n sequence elements in a first similar sequence string to at least n sequence elements in a second similar sequence string, for a first species of the plurality of species; assigns a value to each of n positions of the at least n sequence elements, based upon whether the sequence elements are identical or different in the two similar sequence strings; repeats the comparing and assigning for each species in the plurality of species; sums the values assigned for each of the n positions across the plurality of species; and identifies which of the n positions have the greatest sum value, thereby identifying the positions of conserved difference in the set of similar sequence strings.

21. The computer or computer-readable medium of claim 20, further comprising a database comprising the set of similar sequence strings derived from a plurality of species.

22. The computer or computer-readable medium of claim 20, comprising a neural network.

23. The computer or computer-readable medium of claim 20, comprising a user interface.

24. The computer or computer-readable medium of claim 23, wherein the user interface comprises an input field that permits data entry of the similar sequence strings.

25. The computer or computer-readable medium of claim 23, wherein the user interface comprises a data output file.

26. The computer or computer-readable medium of claim 23, wherein the user interface operates across a network.

27. The computer or computer-readable medium of claim 23, wherein the user interface operates across the internet.

28. The computer or computer-readable medium of claim 23, wherein the user interface comprises a web browser interface.
